(19) United States
(12) Patent Application Publication
(10) Pub. No.: US 2011/0091495 A1
(43) Pub. Date: Apr. 21, 2011

(54) HCV E2 CONSTRUCT COMPOSITIONS AND METHODS

(75) Inventors: Joseph Marcotrigiano, New Brunswick, NJ (US); Jillian L. Whidby, Pilesgrove, NJ (US); Arash Grakoul, Decatur, GA (US)

(73) Assignee: Rutgers, The State University of New Jersey

(21) Appl. No.: 12/988,864

(22) PCT Filed: Apr. 22, 2009
(86) PCT No.: PCT/US09/02502

§ 371 Date: Dec. 14, 2010

Related U.S. Application Data
(60) Provisional application No. 61/046,944, filed on Apr. 22, 2008.

(51) Int. Cl. A61K 39/29; C12N 15/83; C12P 21/02; G01N 33/566; C07K 16/08

(52) U.S. Cl. 424/189.1; 435/320.1; 435/69.1; 424/228.1; 436/501; 530/387.1

(57) ABSTRACT

A construct comprising the ectodomain of the Hepatitis C Virus (HCV) E2 sequence and a mammalian expression system therefor is disclosed. The construct comprises a CMV promoter, prolactin signal sequence, the ectodomain of HCV E2 sequence truncated at aa 664, a thrombin cleavage site and a human Fc domain. The method also relates to an expression system for the construct, which is stably expressed in human embryonic kidney cells 293T. Continuous protein expression in a bioreactor allows for 4 mg of purified protein per liter of cell supernatant.

### CROSS-REFERENCE TO RELATED APPLICATIONS

This application claims priority to U.S. Provisional Patent Application No. 61/046,944 filed on Apr. 22, 2008, the disclosure of which is hereby incorporated by reference in its entirety.

### BACKGROUND OF THE INVENTION

1. Field of the Invention

The invention provides a construct comprising the ectodomain of the Hepatitis C virus (HCV) E2 sequence and a mammalian expression system therefore. More particularly, the invention relates to a construct comprising the CMV promoter, prolactin signal sequence, the ectodomain of HCV E2 sequence truncated at aa 664, a thrombin cleavage site and the human Fc domain. The invention also relates to an expression system for the construct, which is stably expressed in human embryonic kidney cells 293T. Continuous protein expression in a bioreactor allows for 4 mg of purified protein per liter of cell supernatant.

2. Description of the Related Art

Hepatitis C virus (HCV) continues to be a global epidemic. In most cases, HCV infection becomes chronic and can persist for decades, leading to cirrhosis, end-stage liver disease and hepatocellular carcinoma. Currently, 2% of the human population—approximately 123 million people—is infected with HCV. In fact, there are 3-4 times more individuals infected with HCV than HIV, making virus transmission a major public health concern. In the United States, HCV infection is the most common cause of liver transplantation and results in 10,000 to 20,000 deaths a year. There is no vaccine, and current HCV therapy, pegylated interferon-alpha in combination with ribavirin, leads to a sustained response in only 50% of genotype 1-infected patients, the prevalent genotype in the United States. The current HCV treatment stimulates the patient's immune system to clear the virus, but numerous side effects cause many patients to prematurely stop treatment. Given the high prevalence of infection and poor response rate, inhibitors that specifically target HCV proteins with fewer side effects are desperately needed. In addition, an effective vaccine would greatly reduce the spread of the virus.

International Patent Publication No. WO 2008/022401 to Mc Caffrey, et. al, describes preparing an HCV E2 polypeptide having internal deletions of the regions within E2. However, this reference also does not describe a stable cell line that expresses E2. The cells perform transient expression, which is only good for a few days. The E2 DNA is not incorporated into the genome of the cell and after several days, the cells will remove the (gene. This method is inefficient. This publication also does not utilize a construct containing an Fc tag.

U.S. Pat. No. 6,326,171 to Chiron describes preparing an HCV E2 polypeptide involving a specific region of E2 that ends at amino acid 715. The construct used does not contain a tag. The cells used for expression included BSC40 (African Green monkey) and F503 (chimpanzee fibroblasts) are not human cells.

U.S. Pat. No. 6,020,122 to Abbott Laboratories describes preparing an HCV E2 polypeptide without the use of a tag. The cells used for expression are CHO (Chinese Hampster ovaries) cells.

However, there still exists a need in the art for expression of high levels of high quality HCV E2 polypeptides and their uses with HCV in humans, e.g., vaccinations and inhibitors of HCV infection.

### SUMMARY OF THE INVENTION

In a first embodiment, the invention is directed to recombinant HCV E2 ectodomain expression without the production of mostly large, disulfide-bonded aggregates. This process is used to make large quantities of the envelope glycoproteins applicable for a variety of commercial applications including but not limited to: 1) Vaccine design—The recombinant protein can be a vaccine to illicit a strong immune response, protecting individuals from future infection; 2) Therapeutic vaccine—The administration of the protein to patients who are chronically infected with HCV to help the individual develop a more robust immune response either by administration alone or in combination with other medications such as IFN and ribavirin; 3) Diagnostics—enzyme-linked immunoassays can be developed using the purified, recombinant protein to screen patient sera for antibodies against these proteins. Although there are commercial screens currently available for this purpose, the proteins used therein were made in yeast or other expression systems and may not be properly folded and would have different post-translational modifications. Since the present protein is produced in human cells, the post-translational modifications are more similar to those seen on the virus.); 4) Small molecule inhibitors—The ability to make a properly folded E2 could be an important reagent for finding small molecules that bind to E2. (As shown in FIG. 7B, the ectodomain of E2 can bind with high specificity and affinity to a cellular receptor CD81. A similar assay could be used to identify small molecules that prevent this interaction.); and 5) Production of antibodies.

In accordance with the above objects, the invention is directed to a construct comprising the ectodomain of the hepatitis C virus (HCV) E2 sequence and a mammalian expression system therefore comprising the CMV promoter, prolactin signal sequence, the ectodomain of HCV E2 truncated at amino acid 664, a thrombin cleavage site and the human Fc domain. In other embodiments, the construct is specific for the J6 HCV genotype. In other embodiments, the construct is a mutant form of eE2 in which C656 was mutated from a cysteine amino acid to a different amino acid. In other embodiments, the amino acid is serine.

In accordance with the above objects, the invention is also directed to an expression system for a construct of any of claims 1-4, which is stably expressed in human embryonic kidney (HEK) 293T cells.

In accordance with the above objects, the invention is also directed to method of producing HCV eE2 polypeptide comprising: providing a construct comprising: the ectodomain of the hepatitis C virus (HCV) E2 sequence and a mammalian expression system therefore comprising the CMV promoter, prolactin signal sequence, the ectodomain of HCV E2 truncated at amino acid 664, a thrombin cleavage site and the human Fc domain, introducing the construct into HEK293T cells, selection of cells stably expressing the polypeptide, incubating the cells stably expressing the polypeptide in a supernatant, and recovering and purifying the polypeptide from the supernatant. In other embodiments, the method produces about 0.5 to about 15 mg of polypeptide per liter of supernatant. In other embodiments, the method produces about 0.5 to about 4 mg of polypeptide per liter of supernatant. In other embodiments, the method produces about 0.5 to about 2 mg of polypeptide per liter of supernatant. In other embodiments, the method produces the HCV eE2 HCV J6 genotype polypeptide. In other embodiments, the construct is a mutant form of eE2 in which C656 was mutated from a cysteine amino acid to a different amino acid. In other embodiments, the different amino acid is serine. In other embodiments, the incubation occurs in one or more vessels suitable for providing an environment for the cells to express the polypeptide. In other embodiments, the vessels are in a rotating bottle apparatus. In other embodiments, the vessel is a bioreactor.

In accordance with any of the above objects, the invention is also directed to a method wherein the polypeptide is folded and sequesters human HCV receptor sites. In other embodiments, the amount of polypeptide in monomer and dimer form exceeds the amount of polypeptide existing in higher orders, and is recognized by antibodies in the sera of patients infected with HCV. In other embodiments, the polypeptide contains 17 preserved cysteine residues.

In accordance with any of the above objects, the invention is also directed to a method of vaccinating a patient comprising administering to a patient in need thereof, a sufficient amount of a polypeptide produced by the method of any of claims 6-18 to produce a strong immune response protecting the patient from future HCV infection.

In accordance with any of the above objects, the invention is also directed to a method of vaccinating a patient chronically infected with HCV comprising administering to a patient in need thereof, a sufficient amount of a polypeptide produced by the method of any of claims 6-18 to produce a more robust immune response. In other embodiments, an additional therapeutic agent that provides a more robust immune response.

In accordance with any of the above objects, the invention is also directed to a method of inhibiting HCV infection in a human patient comprising administering to a patient in need thereof, a sufficient amount of a polypeptide produced by the method of any of claims 6-18 to effectively block entry into the entry site of human cells. In other embodiments, the sufficient amount of polypeptide is non-toxic to a human patient. In other embodiments, a sufficient amount of an active fragment of the polypeptide is administered.

In accordance with any of the above objects, the invention is also directed to a method for detection of antibodies to HCV in human sera comprising contacting the sera with the polypeptide as prepared herein.

In accordance with any of the above objects, the invention is also directed to a method of producing antibodies comprising introducing the polypeptide as prepared herein to achieve a response that leads to production of antibodies to said polypeptide.

### BRIEF DESCRIPTION OF THE DRAWINGS

FIG. 1. (A) is a diagram showing the organization of the HCV genome showing the 5′ and 3′ NTRs. The open reading frame is represented by the rectangle and is colored light grey for the structural proteins and dark grey for the nonstructural proteins. (B) Polyprotein processing scheme. The black diamonds and open circle denote the cleavage sites for signal peptidase and signal peptide peptidase, respectively. The arrows signify the cleavages performed by the viral encoded NS3-4A (black) and NS2-3 (red). A brief description of each protein is given.

FIG. 2. is a schematic representation of the mammalian expression constructs. Expression is driven by the CMV promoter. The protein consists of an N-terminal signal sequence, the ectodomain of HCV E2, the Fc tag for protein purification and the Fc introns.

FIG. 3. shows expression of the HCV E1 and E2 ectodomains. C-terminal truncations of E1 and E2 were cloned into the mammalian prolactin Fc expression vector and transfected into HEK293T cells. The truncation of each protein relative to the translation start site in core is shown. Supernatants and cells were harvested 72 hours post transfection, separated by reducing (A) and nonreducing (C) SDS-PAGE, transferred to nitrocellulose and probed with an antibody against human Fc. Prolactin was used as a control for protein expression. Panel B is an enlargement of the E1 samples from the reducing gel (A). The cell lysate contains 6 bands, which correspond to basal and 5 additional glycosylation events, while the supernatant contains only the top band.

FIG. 4. shows purification of E2-Fc over protein A resin. Supernatants from cell lines expressing E2-Fc are clarified by centrifugation (load) and applied to the resin. After incubation, the column is extensively washed to remove unbound material (FT). E2 is eluted off the column in five fractions (1-5) by incubation with thrombin protease. The resin before (pre) and after (post) elution are also shown. Samples are analyzed by SDS-PAGE and stained with coomassie Blue.

FIG. 5. shows deglycosylation of eE2 with PNGaseF and Endo H. Purified eE2 was deglycosylated with PNGase F or Endo H under denaturing and reducing conditions and analyzed by SDS-PAGE. The position of the enzymes is also shown.

FIG. 6. shows reducing and non-reducing SDS-PAGE. (A) BHK cells were infected with the wild-type (wt) vaccinia virus strain WR, or recombinant vaccinia viruses expressing eE2 from two HCV strains (Gla or H77), in the presence of [35S]methionine. The radiolabelled proteins from the medium were immunoprecipitated, subjected to 10% SDS-PAGE under reducing and non-reducing conditions. (Image taken from FIG. 3 of Patel et al. 12.) (B) SDS-PAGE of our purified eE2 in the presence and absence of reducing agent and stained with coomassie blue. The samples were run on the same gel with several empty lanes as to prevent reducing agent spread during run running. The intervening lanes are not shown for the sake of saving space. (C) Deglycosylation of eE2 with PNGase F under nondenaturing conditions without reducing agent. The time and incubation temperature is give above. Identical samples were analyzed on SDS-PAGE in the presence and absence of reducing agent and stained with coomassie blue.

FIG. 7. Oligomeric state of eE2. (A) Purified eE2 was applied to Superdex 200 size exclusion column. The arrow denotes void volume where proteins larger than 200 kDa would be expect to elute. The blue (taller peak) and red (shorter peak) lines represent the absorption at 280 and 254 nm, respectively. (B) Enzyme-linked immunoassay for CD81 LEL binding. Tissue culture supernatants of eE2-Fc fusion (no dilution, 1:10 and 1:100 dilutions) were incubated in plates coated with either GST, GST-mouse CD81 LEL, or GST-human CD81 LEL. After washing, bound eE2-Fc was detected with anti-human Fc-HRP. PBS, media from wt HEK293T cells and wells without any coating were used as controls.

FIG. 8. (A) Is the sequence of J6 eE2 (residues 384-664) highlighting the conserved cysteine residues (underlined), and the potential N-linked (Bold) and O-linked (Italics) glycosylation sites. (B) Shows purification of eE2-Fc over protein A-sepharose. Supernatants from cell lines expressing eE2-Fc are clarified by centrifugation (sup loaded) and applied to the resin. After incubation, the column is extensively washed to remove unbound material (flowthrough). E2 is eluted off the column in five fractions (elutions 1-5) by incubation with thrombin protease. The resin before (bound resin) and after elution are also shown (post-cleavage resin). Samples are analyzed by SDS-PAGE and stained with Coomassie Blue.

FIG. 9. (A) Shows deglycosylation of eE2 with PNGaseF and Endo H. Purified eE2 was deglycosylated with PNGase F or Endo H under denaturing and reducing conditions and analyzed by SDS-PAGE. The position of the enzymes and eE2 are also shown. (B) Shows mapping the N-linked glycosylation sites. The ten panels contain LC-MS data, corresponding to the peptides containing the 11 N-linked glycosylation sites. Note that one peptide contains two glycosylation sites. The top spectra are for glycosylated peptides, while the bottom spectra are for peptides deglycosylated with PNGase F. The height of the peak corresponds to relative abundance. The peptide sequence and measured molecular weights are given.

FIG. 10. (A) Shows SDS-PAGE analysis of purified eE2 in the presence and absence of β-mercaptoethanol (β-ME) and stained with Coomassie blue. (B) Is a graphical depiction of purified eE2 applied to a Superdex 200 size exclusion column equilibrated with 50 mM HEPES pH 7.5, 150 mM KCl, 5% glycerol. The arrows denote the position of the void, dimer, and monomer.

FIG. 11. Shows differential labeling of free and disulfide-linked cysteines. Free and disulfide bonded cysteines were labeled with NEM (an addition of 57Da) and IAM (an addition or 125Da), respectively. LC-MS data for peptides containing C656 (A) and C459 (B) are shown. The top spectra correspond to labeling with NEM and the bottom spectra with IAM. C656 is free, while C459 is found in a disulfide bond. All of the other cysteine residues were labeled with IAM (data not shown), suggesting the formation of eight disulfide bonds. (C) Is a graphical depiction of purified eE2-C656S applied to a Superdex 200 size exclusion column equilibrated with 50 mM HEPES pH 7.5, 150 mM KCl, 5% glycerol. The arrow denotes the position of the void, dimer, and monomer.

FIG. 12. Shows size exclusion chromatography of eE2 and eE2-C656S at pH7 and pH5. eE2 samples were applied to a Superdex 200 column equilibrated with 25 mM sodium phosphate pH 5.0 or 7.0, and 50 mM KCl. The location of void, dimer and monomer are noted.

FIG. 13. Shows analytical ultracentrifugation data for eE2-C656S at pH7 and pH5. Two-dimensional spectrum/Monte Carlo analysis of HCN sedimentation velocity data. Measurements of eE2 were made at low concentration (0.25 OD₂₃₀) at pH 5 (A) and (pH 7) (B), and at higher concentration (0.8 OD₂₃₀) at pH 5 (C) and pH 7 (D). All samples show the presence of monomer and dimer. The pH 7 samples show the presence of a trimer species. Heterogeneity in shape and molecular weight is more prominent in the pH 7 samples for both monomer and dimer species. Larger species appear more globular than smaller species according to the frictional coefficients. The units of the color gradient are in OD₂₃₀. (E) Integral van Holde-Weischet distributions from sedimentation velocity experiments with HCV to test pH reversibility. Shown is the distribution for HCV at pH 5 (grey squares), at pH 7 (blue triangles) and at pH 7 after buffer exchange from pH 7 to pH 5, and then back pH 7 (red circles). The distributions all show about 10% high-molecular weight aggregate, as well as about 50% of the protein sedimenting at the same speed. The remaining 40% of the protein sediments slightly faster for samples at pH 7, indicating the presence of higher molecular weight species. Importantly, the sample which has undergone dialysis from pH 7 to pH 5 and then back to pH 7 shows that most of the material has reverted back to the distribution seen for the pH 7 sample that was never exposed to pH 5.

FIG. 14. Is a graphical depiction of circular dichroism spectroscopy of eE2 and eE2-C656S at pH 7 and pH 5. CD spectra are shown as millidegrees versus wavelength (nm). Error bars for each data point are given.

FIG. 15. Shows graphical depictions of functional analyses of eE2 and eE2-C656S. (A) ELISA plates were coated with eE2 and probed with a series of ten fold dilutions of serum from patients infected with HCV (genotypes 1, 2, or 3) and healthy donor. Antibodies in HCV-infected patient sera could detect eE2 up to 1:100,000 dilution. (B) Cells were incubated with HCVcc plus GST, GST-mCD81 LEL, GST-hCD81 LEL, eE2, and eE2-C656S. Three days post-infection the cells were fixed, focus forming units were determined, and percent of inhibition was calculated. eE2, eE2-C656S, and hCD81 inhibit HCVcc infection. Error bars represent standard error of the mean for two independent experiments. Each experiment was performed in duplicate. (C) Cells were incubated with eE2, GST and GST-hCD81-LEL at three concentrations (200, or 100 μg/mL). Three days later, cells were analyzed for viability using flow cytometry. The results demonstrate that eE2 is not toxic when applied to cells at the concentration that inhibits HCVcc infection. (D) Enzyme-linked immunoassay for CD81-LEL binding. Tissue culture supernatants of eE2-Fc fusion (no dilution, 1:10 and 1:100 dilutions) were incubated in plates coated with either GST, GST-mouse CD81-LEL, or GST-human CD81-LEL. After washing bound eE2-Fc was detected with anti-human Fc-HRP. PBS, media form wt HEK293T cells and wells without any coating were used as controls. Both eE2 and eE2-C656S bound to only human CD81.

### DETAILED DESCRIPTION OF THE INVENTION

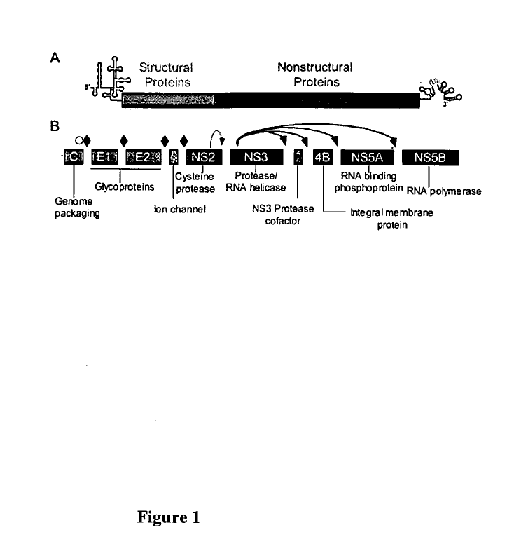

HCV is a member of the family Flaviviridae, which also includes Pestiviruses and Flaviviruses. Since its identification in 1989, phylogenetic analysis of various isolates has resulted in the classification of six distinct genotypes that are further divided into a number of subtypes (e.g. 1a, 1b, 1c, etc.). The HCV virion consists of an enveloped nucleocapsid containing the viral genome, a single-stranded, positive sense RNA that encodes a single, open-reading frame (FIG. 1).

Once the virus penetrates a permissive cell, the HCV genome is released into the cytosol where the viral RNA is translated in a cap-independent manner by an internal ribosome entry site (IRES) located within the 5′ nontranslated region (NTR). Translation generates a viral polyprotein that is proteolytically processed by cellular and viral encoded proteases into ten proteins (FIG. 1). The N-terminal region is cleaved by cellular signal peptidase and signal peptide peptidase to yield the structural components of the virus particle (Core, envelope proteins E1 and E2) and an ion channel (p7). The mature nonstructural proteins (NS2, NS3, NS4A, NS4B, NS5A, and NS5B) are liberated by two essential viral enzymes: the NS2-3 cysteine protease and the NS3-4A serine protease. NS3-5B comprise the minimal viral proteins necessary to form the RNA replication machinery or replicase. HCV replication occurs in association with the perinuclear and ER membranes, utilizing both cellular and viral proteins. Replication involves the synthesis of a genome-length, minus strand that serves as a template for the production of new positive strands for packaging. Not much is known about HCV assembly and egress, since a system to study these processes has not been available until recently, however extrapolations have been made from comparison with other flaviviruses. HCV virion assembly is thought to occur on the ER membrane. Newly synthesized, genomic RNAs are encapsulated by core. These nucleocapids bud into the ER, encircling it with the envelope membrane and HCV glycoproteins. The virions travel through the secretory pathway and are released at the cell membrane.

Construct Design, Expression and Purification of HCV eE2

HCV E2 is a type I transmembrane protein with an amino-terminal ectodomain and a carboxy-terminal membrane-associating region. The protein is composed of 333 residues. E2 is glycosylated and contains intramolecular disulfide bonds, making it extremely challenging for structural, biochemical, and biophysical studies. However, we use E2 ectodomain (eE2) (amino acids 384-660 of the HCV polyprotein genotype 1a starting from position 1 in the HCV core and/or amino acids 384-664 of the HCV polyprotein genotype 2a) that is lacking the C-terminal membrane anchor, since this has been demonstrated to express well¹¹ and retain interactions with CD81 and SR-BI.

Biochemical, biophysical, and structural studies rely on the production of purified eE2. Studies on recombinant E1 and E2 expression have yielded two different forms of the molecules: a glycosylated protein with intramolecular disulfide bonds that is believed to be the active form and high molecular weight aggregates caused by intermolecular disulfide bonds. The formation of disulfide bonded aggregates and misfolded protein has hampered structural and biophysical studies on the HCV glycoproteins. Our approach was to explore three different variables (cell type, genotype and affinity tag) to determine which construct produced high levels of properly folded eE2.

Cell Type

The cell lines used for expression can have a pronounced effect on stability. Expression of viral glycoproteins in bacteria has proven to be a challenge and several reports document that expression of eE2 in E. coli leads to the formation of insoluble, inclusion bodies. This is not surprising given the large number of potential glycosylation sites and intramolecular disulfide bonds. Proteins with these posttranslational modifications are known to be difficult to express in bacteria. The unique properties of the mammalian cellular environment make this the ideal choice for homologous expression of eE2. HEK293T and Huh7 cells were the first cell lines tested, since these have been shown to produce active E1 and E2 in the form of HCVpp and HCVcc. Initially, HEK293T were chosen over Huh7 owing to ease of handling, robust growth rate, excellent transfectability, and high capacity for recombinant protein expression.

Genotype

Given that E2 forms the virus particle, the surface of the glycoproteins would be expected to undergo mutation to escape immune pressure; therefore the behavior of E2 from different genotypes might be markedly different. In fact, it has been shown that preparations of E2 from different genotypes can vary in binding to CD81 and the aggregation state of the glycoproteins. This approach of expressing the same protein from different genotypes was essential to defining constructs that would yield the highest amount of folded protein for functional studies. In fact the same protein from different genotypes behaved differently in terms of amount expressed, solubility and protein stability.

Affinity Tag

The addition of an affinity tag can permit rapid purification and added protein stability. In previous work relating to the expression and purification of eE2, a construct consisting of the E1 signal sequence followed by the ectodomain of E2 and a C-terminal, six histidine tag was made and expressed in HEK293T cells. The signal sequence would target eE2 to the ER where glycosylation and disulfide formation occur naturally and permit the protein to be secreted into the media. Expression levels were very low and supernatants stripped the Ni from the resin, resulting in poor retention of the eE2. Buffer exchange of the media inhibited the Ni stripping while promoting eE2 binding, however this approach would not be applicable for large-scale expression. Therefore, a better tag was needed which had high affinity and specificity to a resin, and could be used in the presence of media.

We sought to use the Fc domain of human IgG as an affinity tag. The Fc domain is glycosylated, contains an intermolecular disulfide bond, is very soluble, binds with high affinity to protein A resin, and can bind to the resin in the presence of tissue culture media. Our hypothesis was that since the Fc domain is glycosylated and disulfide bonded it might assist similar modifications found on eE2 and only a properly folded Fc domain is competent for secretion and protein A binding. These unique properties of the Fc domain seemed ideal for this situation.

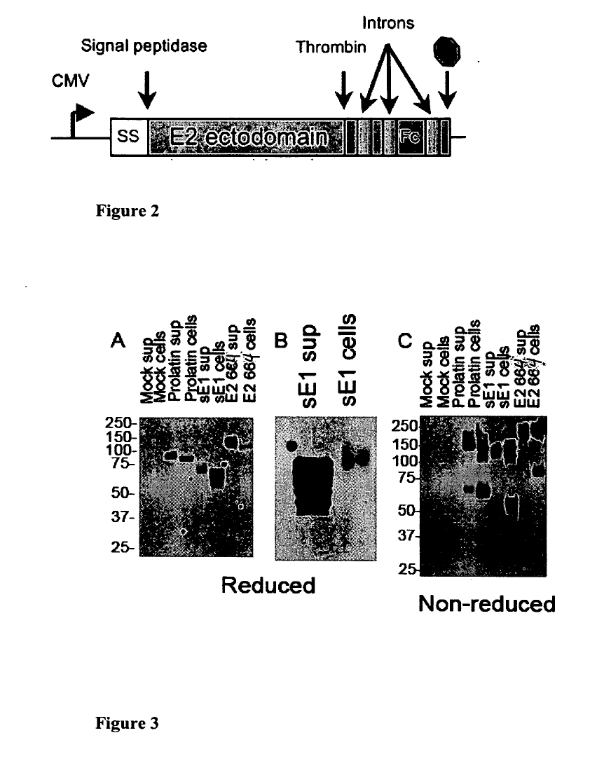

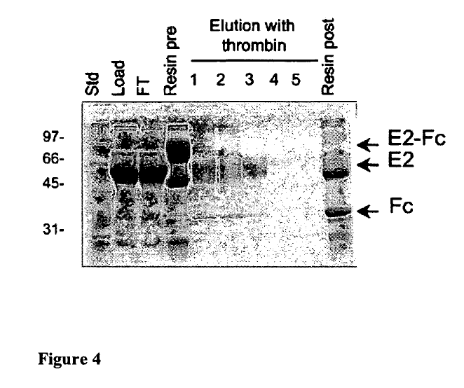

The HCV eE2 was cloned as a fusion with the Fc domain into a modified pcDNA3.1 vector for constitutive expression in mammalian cell lines. A schematic representation of the expression construct is shown in FIG. 2. Amino-terminal to the gene of interest is the prolactin signal sequence that targets the ectodomains to the endoplasmic reticulum followed by a short linker region to allow for efficient cleavage by signal peptidase. On the carboxy-terminus is a thrombin cleavage site followed by the Fc domain to facilitate protein purification. The Fc domain construct contains several natural introns, which has been shown to increase protein expression by several fold. Initially, we constructed eE2 from genotypes 1a (H77) (amino acids 384-661) and 2a (J6) (amino acids 384-664) into the plasmids described above and expressed in HEK293T cells. The discrepancy between the numbers is due to a small insertion into HVR2 region in J6. These two genotypes were chosen since they have been demonstrated to be infectious in chimpanzees. The constructs were transiently transfected into HEK293T cells. Seventy-two hours posttransfection the cells and supernatants were harvested, separated by reducing (FIG. 3A) and nonreducing SDS-PAGE (FIG. 3C), and probed with anti-human Fc antibodies. Prolactin was used as a control and the ectodomain of HCV E1 was also expressed in this system. The E1 and E2 ectodomains yielded good expression levels without the production of disulfide-bonded aggregates, as determined by reducing and nonreducing SDS-PAGE (FIG. 3). For the E1 ectodomain the cell lysates contain six bands, which we believe correspond to 0-5 glycosylation events (FIG. 3B). This result is consistent with the prediction that eE1 contains 4 glycosylation sites and the Fc an additional site. The eE1 supernatants contain only a single band that migrates slightly slower than the top band seen in the cell lysate, suggesting that the eE1-Fc fusion has been fully processed and secreted through the secretory pathway. This result supports our hypothesis that the Fc domain might promote protein modification and secretion. When the identical samples were analyzed under nonreducing SDS-PAGE (FIG. 3C), the cell lysates from all constructs contain a faster and slower migrating band relative to reduced form (compare similar lanes in FIGS. 3A and 3C). The Fc tag is an intermolecular, disulfided-bonded dimer while the HCV glycoproteins contain intramolecular, disulfide bonds. Therefore, the faster migrating band corresponds to a monomeric protein with intramolecular disulfide bonds, while the slower migrating band is a dimer stabilized by the intermolecular disulfide bond in the Fc tag. The slower migrating band is approximately double the molecular weight of the reduced form consistent with an intermolecular disulfide bond between two Fc molecules. Only the slower migrating band is present in the supernatant of the cells transfected with the HCV glycoproteins, suggesting that only those proteins with a fully formed Fc dimer are secreted. The supernatants from the prolactin control cells do show both bands. However, this could be due to the noticeable cell death that occurred with that population. The presence of the disulfide-bonded homodimer in supernatant supports our hypothesis that only a properly folded Fc domain can be secreted.

Encouraged by our preliminary expression results, we attempted to produce a stable cell line that constitutively secreted eE2. HEK293T cells were transfected with eE2 constructs from both genotypes and were placed under hygromycin B selection. We developed a quantitative enzyme-linked immunosorbent assay (ELISA) against the Fc domain to quickly identify and quantitate which drug resistant cells were expressing the most eE2. We have isolated HEK293T cells that constitutively express eE2 from J6 and H77 at levels comparable to transient transfection.

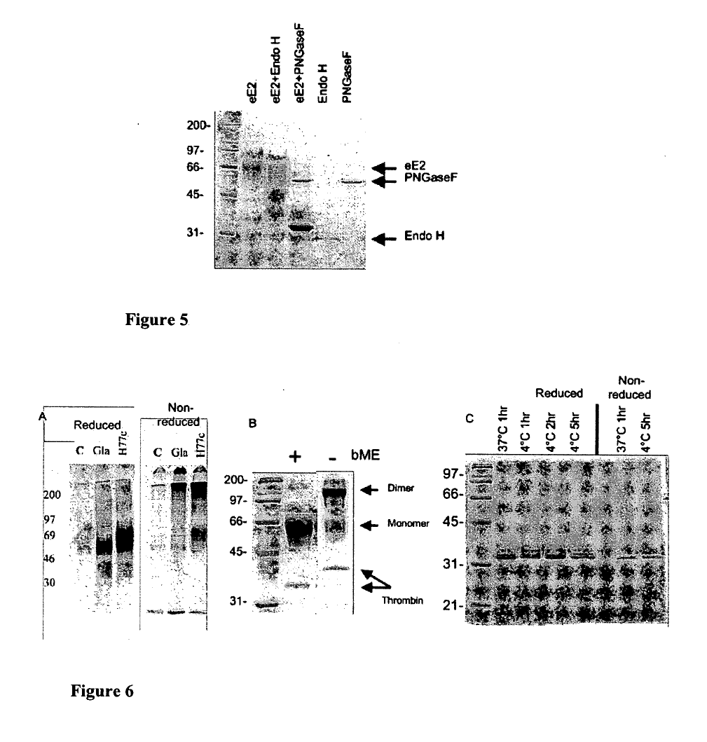

With the creation of a stable cell line expressing eE2, we set out to determine purification conditions. The stable eE2 cell lines were expanded into 10 roller bottles with media containing fetal bovine serum to assist in cell attachment and growth. Once the cells become confluent, the supernatant is harvested, replaced with media without serum, and left for another two days. The media from both harvests is pooled together (approximately 1 L total volume) and incubated in the presence of protein A resin with agitation overnight in a cold room. The next morning the resin is harvested and washed extensively with buffer. The fusion protein can be eluted off the resin by either the addition of thrombin to cleave between eE2 and the Fc or by lowering the pH of elution buffer to disrupt the Fc/protein-A interaction. Since HCV and other viruses undergo a low pH triggered membrane fusion, eluting eE2 by low pH may cause a structural rearrangement in the glycoproteins. The resin can be washed with buffer to collect eE2, leaving the contaminants bound to the resin. Samples (5 μL) of each step of the purification are analyzed by SDS-PAGE and stained with coomassie blue protein dye (FIG. 4). The presence of J6 eE2 was confirmed by a combination of N-terminal sequencing and tryptic digestion followed by mass spectrometry. The final protein yield is about 0.5-1 mg of eE2 per liter of supernatant in one preferred embodiment, in other embodiments, the yield is about 2 mg/liter, in other embodiments, the yield is about 4 mg/liter and in still other embodiments, the yield is about 15 mg/liter.

Properties of J6 eE2 Glycosylation

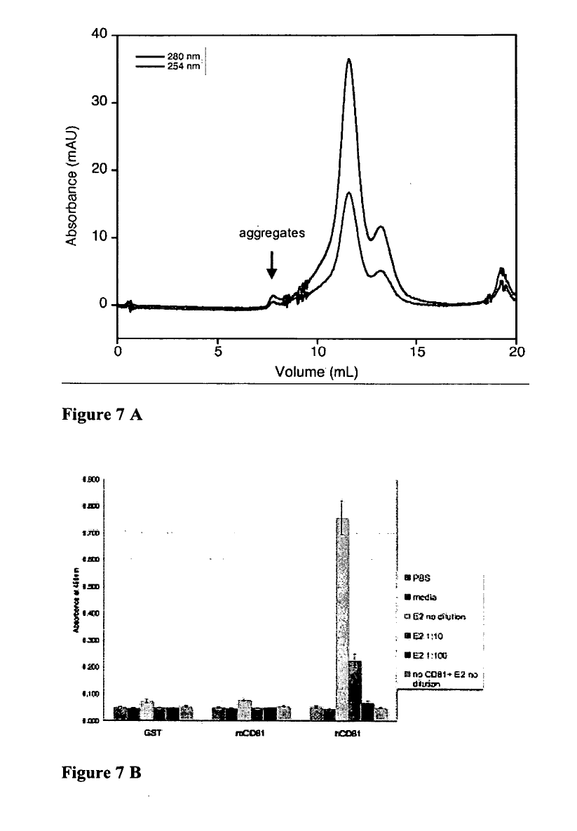

The addition and modification of glycans onto proteins is one of the major biosynthetic pathways found in the lumen of the ER. During translation of a glycoprotein, an oligosaccharide composed of two N-acetylglucosamine, nine mannose, and three glucose molecules are transferred en bloc to a sequon of Asn-X-Ser/Thr (where X is any amino acid except Pro). Oligosaccharyl transferase (also known as N-acetylglucosaminyltransferase), is an ER membrane bound enzyme that characterizes the transfer to the NH₂ group of the Asn side chain. However, not every sequon is modified and the efficiency of transfer can vary for the same sequon, leading to a mixture of modified and unmodified protein. Once the oligosaccharide is on the protein, it can be trimmed and further glycans added as the protein travels through the ER and Golgi apparatus. The processing pathway is highly ordered and begins in the ER with the removal of all the glucose and certain mannose molecules. The remaining steps occur in the Golgi apparatus, where three more mannose molecules are removed and various sugars are added. Although the steps of processing and subsequent sugar addition are rigidly ordered, complex oligosaccharides can be heterogeneous. The end result is two broad classes of N-linked oligosaccharides, referred to as complex and high mannose oligosaccharides. Whether a given oligosaccharide remains high-mannose or is processed is largely determined by its configuration on the protein and if the site is accessible to the modifying enzymes. High mannose and complex oligosaccharides can be differentiated by endoglycosidase H (Endo H) sensitivity, since Endo H will only cleave high mannose glycans. Peptide-N-glycosidase F (PNGase F) will remove all types of N-linked glycosylation. eE2 appears as a smeary band by reducing SDS-PAGE (FIG. 4), which is consistent with what is seen with other glycosylated proteins. To confirm that the eE2 is glycosylated, the protein was denatured in the presence of SDS and reducing agent (dithiothreitol DTT), and incubated with endoglycosidases, Endo H or PNGase F (FIG. 5). PNGase F collapses the protein from 66 kDa to about 35 kDa. Each glycosylation event would increase the protein's molecular weight by about 2-2.5 kDa. The difference in molecular weight seen in the presence and absence of PNGase F can be explained if all 11 putative glycosylation sites are modified, resulting in an increase of approximately 22-27.5 kDa. Since eE2 is expressed by secretion into the media, the glycans would be predicted to be complex carbohydrates and insensitive to Endo H digestion. FIG. 5 documents that eE2 is mostly insensitive to Endo H treatment, consistent with its mode of expression.

Disulfide Bond Formation and Aggregation

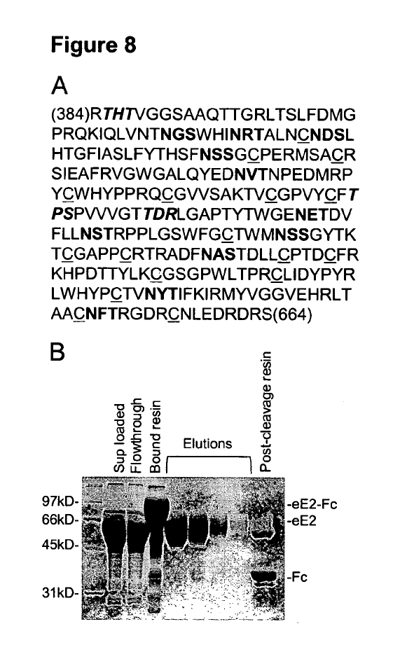

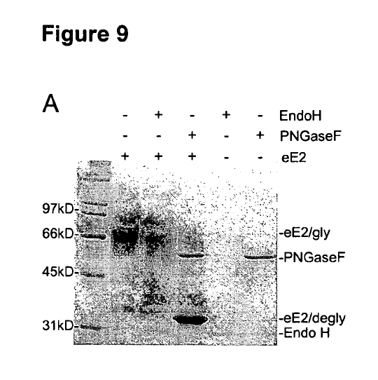

As mentioned previously, expression of HCV eE2 has resulted in the formation of high molecular weight aggregates caused by the presence of intermolecular disulfide bonds that are considered to be misfolded. FIG. 6A displays what is commonly seen for eE2 expression in the presence and absence of reducing agent (image taken from FIG. 3 of Reference 12). Nonreducing SDS-PAGE demonstrated that our purified, glycosylated eE2 was mostly dimer and monomer with higher order aggregates to a lesser extent. Our result is in contrast to what has been published previously, which has shown the E2 is mostly monomer and high molecular weight aggregate under non-reducing conditions (compare FIGS. 6A and 6B). The smeary nature of eE2 made molecular weight determination difficult. So we decided to deglycosylate eE2 with PNGaseF under native conditions and then analyze the product by nonreducing, SDS-PAGE, which would allow for sharper bands and better MW estimations (FIG. 6C). Surprisingly, the natively deglycosylated protein appears to be monomeric by nonreducing SDS-PAGE. We confirmed that the PNGase F preparation was purified in the absence of any reducing agent (New England BioLabs). To further characterize the oligomeric state of glycosylated eE2, the protein was subjected to gel filtration chromatography (Superdex 200 column, GE Healthcare) (FIG. 7A). eE2 appears mostly as a single species (estimated to be 75% by peak area) with a smaller second peak as a shoulder after the main peak. There is an extremely small peak in the column void volume, which would represent oligomers with MW greater than 200 kDa (denoted with an arrow). Proteins of defined MW were applied to the gel filtration column under identical buffer conditions and the MWs of the two main peaks were calculated to be 123 kDa and 75 kDa. These MWs would be consistent with a dimer and monomer, assuming that the fully deglycosylated monomer is less than 66 kDa (as determined by SDS-PAGE). There is a striking similarity between the ratio of dimer to monomer in the gel filtration data and in non-reduced SDS-PAGE. eE2 has 17 highly conserved cysteine residues, which could result in the formation of eight disulfide bonds. It is possible that in the absence of reducing agent the protein does not completely unfold and some structure remains in non-reducing SDS-PAGE. It has been shown that aggregated eE2 will not bind to CD81¹². We performed an enzyme-linked immunoassay to test binding of eE2 to the LEL from mouse and human CD81 (FIG. 7B). The supernatants from our HEK293T cells that express eE2 showed strong and specific binding to only human CD81 and almost no binding to mouse CD81. This species-specific binding to CD81 is consistent with what has been seen previously⁵.

### DESCRIPTION OF PREFERRED EMBODIMENTS

The following examples are meant to illustrate, but not limit the scope of the invention.

### Example 1

Production of eE2 Stable Cell Lines. HEK293T cells were cultured in Dulbecco's Modified Eagle Medium supplemented with 10% FBS (DMEM10). A 6-well plate was seeded with 0.5×10⁶ cells per well and the pPro-eE2-Fc vector (from J6 strain) was transfected the following day using FuGene-HD (Roche Diagnostics, Basel, Switzerland). After three days, the cells were placed under hygromycin (Calbiochem, San Diego, Calif.) selection at 75 μg/ml. Individual colonies were selected, expanded, and tested for eE2-Fc expression using an anti-Fc ELISA.

### Example 2

ELISA for eE2-Fc. MaxiSorp plates (Nunc, Thermo Fisher Scientific, Rochester, N.Y.) were coated with 100 μL of supernatant for two hours at room temperature. The wells were washed 3× with 200 μL of PBS+0.05% Tween-20 (PBS-T), then blocked with 200 μL of 2% BSA in PBS-T for one hour at room temperature. After three more washes with PBS-T, 100 μL of goat anti-Fc antibody (Pierce, Thermo Fisher Scientific, Rochester, N.Y.) at 1:15,000 dilution (in PBS-T) was incubated for one hour at room temperature. The ELISA was developed with TMB substrate (Pierce) and quantified using the SpectraMAX 250 plate reader and SOFTMax 2.6 software.

### Example 3

Expression and Purification of eE2. The supernatant from stable cell lines of Example 1 was harvested, centrifuged to remove cellular debris, and filtered through a 0.22 μm membrane. The eE2-Fc protein was applied to protein A-conjugated resin (GE Healthcare, Piscataway, N.J.) overnight with gentle rocking. The resin was pooled together the next day, washed with buffer (50 mM HEPES pH 7.5, 150 mM KCl, 5% glycerol), and incubated with thrombin protease (GE Healthcare, Piscataway, N.J.) to release the protein from the Fc tag. After cleavage, the protein eluate was consolidated and the concentration determined by Bio-Rad Protein Assay. The protein was analyzed by SDS-PAGE and Coomassie staining. Yields are found to be about 2 mg of eE2 per liter of supernatant.

### Example 4

Deglycosylation of eE2. eE2 was deglycosylated using either Endo H or PNGase F according to manufacturers protocol (New England Biolabs, Ipswich, Mass.). 20 μg of eE2 was denatured and 10 Units of EndoH or PNGaseF was added. The reaction was incubated at 37° C. for one hour and analyzed by SDS-PAGE followed by Coomassie staining.

### Example 5

Mapping the N-linked Glycosylation Sites. The eE2 protein sample was denatured in 6M urea, then reduced with 10 mM DTT for 30 min at 60° C. After denaturation, 20 mM iodoacetamide was added to alkylate sulfhydryl groups and incubated in the dark for one hour at room temperature. Following this treatment, the sample was buffer-exchanged into 50 mM NH₄HCO₃. The sample was digested using either sequencing grade trypsin (Promega, Madison, Wis.) or chymotrypsin (Roche Diagnostics) according to manufacturers' protocol. Digested samples were dried via speed vacuum and reconstituted in 50 mM NH₄HCO₃. Deglycosylation with 50 U of PNGaseF (New England Biolabs, MA) was incubated for 1 hour at 37° C. and the reaction was stopped with 0.1% trifluoroacetic acid (TFA).

### Example 6

Liquid Chromatography/Mass Spectrometry Analysis

All LC-MSMS experiments were performed using the U3000 (Dionex, Sunnyvale, Calif.) in nano-LC mode on line with LTQ (Thermo Fisher Scientific). Samples were first solubilized in 0.1% TFA and loaded onto a 75 μm×12 cm emitter column self-packed with Magic C18AQ, 3 μm 200 Å (Michrom Bioresources Inc, Auburn, Calif.). The sample was eluted using a linear gradient from 98% of 0.1% formic acid in water to 45% of 0.1% formic acid in acetonitrile over 30 min. Mass spectrometry data was acquired using a data-dependent acquisition procedure with a full scan cyclic series. This was followed by zoom scans and MSMS scans of the five most intense ions with a repeat count of two and a dynamic exclusion duration of 60 sec.

The LC-MSMS data was searched against a human database using a local version of the Global Proteome Machine (GPM USB, Beavis Informatics Ltd, Winnipeg, Canada). Carbamidomethylation of cysteine was used as the fixed modification, while oxidation of methionine and deamination of asparagine were used as potential modifications. Manual interpretation and peak integration was performed on all peptide peaks covering potential glycosylation sites (NXT/S).

### Example 7

Gel Filtration Analysis of eE2. Purified eE2 protein was loaded onto a Superdex200 10/300 size exclusion column (GE Healthcare) equilibrated with either HEPES buffer (50 mM HEPES pH 7.5, 150 mM KCl, 5% glycerol) or phosphate buffer (25 mM sodium phosphate pH 5.0 or 7.0, 50 mM KCl).

### Example 8

Free Cysteine Analysis To label free cysteines, the protein sample was incubated with a 20-fold molar excess of N-ethylmaleimide (NEM) and 6M guanidine-HCl at room temperature for one hour in the dark. The sample was then buffer exchanged to 6M guanidine-HCl using a spin filter and washed three times with 400 μl of 6M guanidine-HCl to remove the NEM. Disulfide bonds were reduced by adding 10 mM DTT at 60° C. for 30 min. The newly generated free sulfhydryl groups were alkylated with 20 mM iodoacetamide (JAM) at room temperature for one hour in the dark. After buffer exchange into 50 mM NH₄HCO₃, the samples were digested with trypsin protease at 37° C. overnight. The samples were then deglycosylated with PNGaseF (100 U) at 37° C. for three hours and acidified prior to LC-MSMS analysis.

The LC-MSMS data was searched using Sequest against an E. coli genome database (a common contaminant of in-gel digest) and added sequences of the target protein. +57Da (alkylation by IAM) and +125Da (alkylation by NEM) on cysteine, oxidation of methionine (+16Da), and deamination of asparagine (+1Da) were used as potential modifications. The identification was confirmed manually.

### Example 9

Analytical Ultracentrifugation All sedimentation experiments were performed with a Beckman Optima XL-I at the Center for Analytical Ultracentrifugation of Macromolecular Assemblies at the University of Texas Health Science Center at San Antonio. Sedimentation velocity data were analyzed with UltraScan version 9.9. All measurements were made at 230 nm in intensity mode, at 20° C., and at 37 krpm, using standard upon 2-channel centerpieces. All samples were measured in 25 mM sodium phosphate buffer containing 50 mM KCl, adjusted to either pH 5.0 or 7.0. Concentration dependency of the sedimentation data was assessed by sedimenting the sample at both high concentration (˜0.8 optical density (OD) at 230 nm) and at low concentration (˜0.25 OD at 230 nm). Hydrodynamic corrections for buffer density and viscosity were made according to methods outlined in Laue et al. and as implemented in UltraScan. The data were analyzed by 2-dimensional spectrum analysis (2DSA) using the ASTFEM-RA solution with simultaneous removal of time-invariant noise. Molecular weight and shape distributions obtained in the 2DSA were further refined by Monte Carlo analysis. Composition comparisons were made by overlaying sedimentation coefficient distributions derived from the van Holde-Weischet analysis. The calculations were performed on the Lonestar cluster at the Texas Advanced Computing Center at the University of Texas at Austin, and at the Bioinformatics Core Facility at the University of Texas Health Science Center at San Antonio.

### Example 10

Circular Dichroism The protein sample was buffer-exchanged into 25 mM sodium phosphate pH 5.0 or 7.0, and 50 mM KCl. The CD spectra in the wavelength range of 195-260 nm were measured at 0.5 nm intervals on an Aviv spectropolarimeter model 400 (Lakewood, N.J.) at 25° C. A quartz cell with a path length of 0.1 cm was used. The CD spectra were analyzed for secondary structure using multilinear regression as described previously.

### Example 11

eE2 ELISA using Human sera 96-well EIA/RIA plates (Corning, Lowell, Mass.) were coated with 100 μl of a 1 μg/ml solution of eE2 in NaHCO₃ overnight at 4° C. The plates were washed twice with 200 μl/well PBS-T, then blocked with a 10% solution of normal goat serum in PBS-T (Jackson ImmunoResearch, West Grove, Pa.) for one hour at 37° C. Human serum was isolated from whole blood samples (Emory University School of Medicine, PI Arash Grakoui, IRB#1358-2004) collected in SST tubes (Becton Dickenson, Franklin Lakes, N.J.) via centrifugation and frozen in aliquots at −80° C. Ten-fold serial dilutions were made for each serum sample using binding buffer composed of 0.1% normal goat serum in PBS-T. 100 μl of the dilutions was added to each well of the plates and incubated for 90 minutes at room temperature. The plates were washed eight times with PBS. 100 μl of goat anti-human IgG-Biotin conjugate (Biosource, Camarillo, Calif.) diluted 1:20,000 in binding buffer was added and allowed to incubate for 90 minutes at room temperature. Finally, 100 μl streptavidin-HRP conjugate (Biosource) was added to each well at a 1:2,000 dilution and incubated for 45 minutes at room temperature. Using TMB substrate solution (Ebioscience, San Diego, Calif.), absorbance was measured using a VersaMax Microplate reader and SoftMax Pro software (Molecular Devices, Sunnyvale, Calif.).

### Example 12

HCVcc Infection in the presence of purified proteins. Approximately 100 TCID50 of Cp7 viruses were incubated with two-fold dilutions of the purified eE2, eE2-C656S, GST, GST-CD81LEL or GST-mCD81 starting at 2004 ml. 6.0×10³ cells were seeded into a collagen-coated 96-well plate. The virus-protein mixture was incubated with the cells for three days at 37° C. Cells were stained by immunohistochemistry as previously described.

### Example 13

Cytotoxicity. Huh-7.5 cells were incubated with various dilutions of the purified proteins as described above. Three days later, cells were washed twice with PBS, harvested by trypsinization, and resuspended in 100 μl of PBS. Cells were stained with BD Via-Probe™ (BD Biosciences, San Jose, Calif.) according to the manufacturer's instructions and counted using FACSCalibur (BD Biosciences) equipment and FlowJo (v8) analysis software.

### Example 14

Expression and Purification of GST and GST-CD81-LEL. CD81-LEL was expressed with an amino-terminal GST tag and carboxy-terminal histidine tag. The protein was expressed and purified as described previously. The GST tag alone was expressed and purified using the same method.

Results

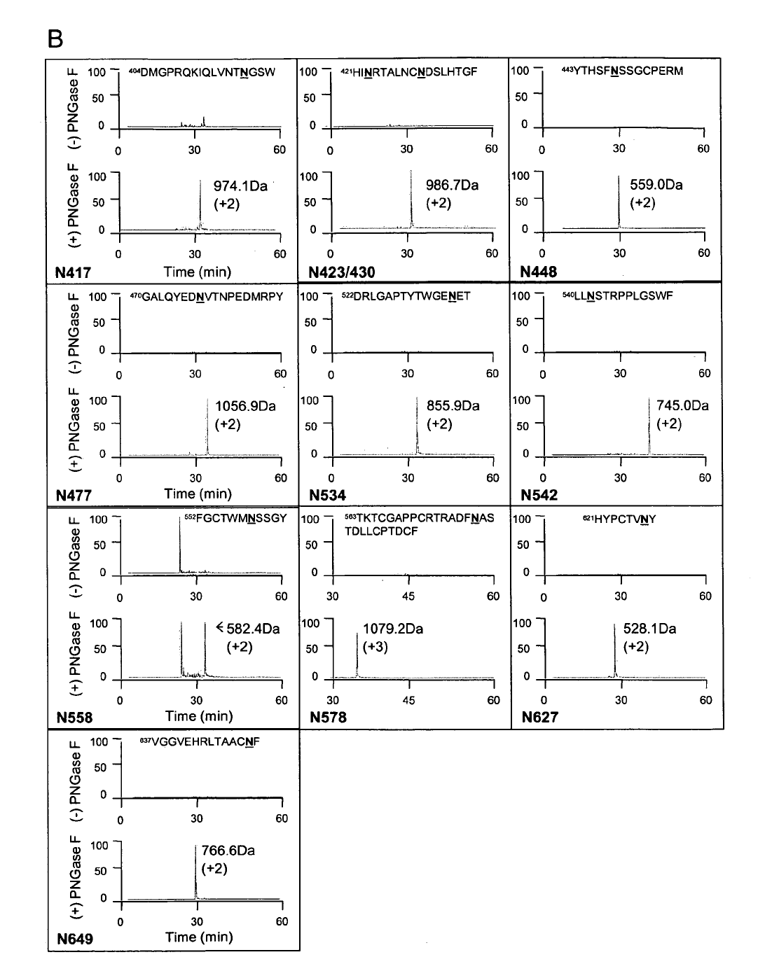

Expression of eE2 in E. coli, yeast, insect cells, CHO cells, and various other eukaryotic and viral recombinant systems, has consistently resulted in the formation of insoluble, misfolded and aggregrated protein. We sought to develop a system for the expression of HCV eE2 that would yield large amounts of highly purified, active protein for functional studies. Our approach was to utilize cell lines that have been shown to produce functional E2, while adding an affinity tag to increase eE2 stability and facilitate purification. HEK293T cells were chosen owing to a) their ability to produce functional E2 in the form of HCV pseudoparticles (HCVpp), b) their ease of handling and robust growth rate and, c) their excellent transfectability and high capacity for recombinant protein expression. We expressed the J6 (genotype 2a) E2 ectodomain (aa384-664) because this fragment of E2 has been shown to be the minimal functional unit for binding and entry (FIG. 8A). eE2 is preceded by a prolactin signal sequence and signal peptidase cleavage site to promote trafficking through the secretory pathway and followed by a thrombin cleavage site and Fc tag (eE2-Fc). The Fc tag was chosen since it is glycosylated and disulfide-bonded, which may assist similar posttranslational modifications on eE2. We have created a HEK293T cell line that stably secretes eE2-Fc into the media. The eE2-Fc was isolated using protein A resin and eE2 was subsequently separated from the Fc tag via thrombin protease cleavage, leaving the Fc tag bound to the resin (FIG. 8B). The calculated molecular weight of the J6 eE2 protein is 33 kDa, although it migrates around 60 kDa by reducing SDS-PAGE. This molecular weight discrepancy and the diffuse nature of the band are observations consistent with glycosylated proteins.

### Example 15

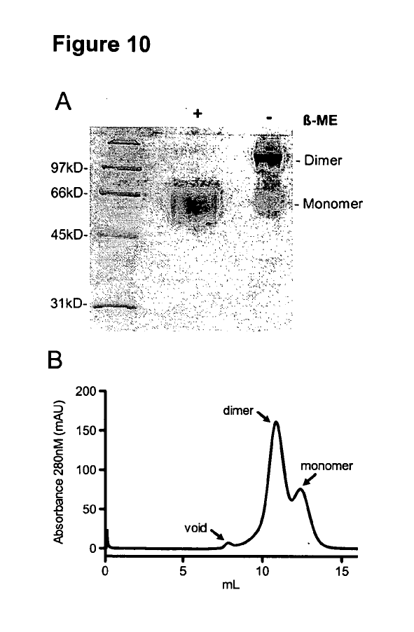

Analysis of glycosylation. Glycosylation of viral envelope proteins is critical for folding, structural integrity, and immune evasion. The number and conservation of glycosylation sites varies across different HCV genotypes. J6-E2 contains 11 potential N-linked glycosylation sites (N-X-T/S) along with three potential O-linkage consensus sites (FIG. 8A). We investigated the extent of eE2 N-linked glycosylation and the type of oligosaccharide at each site using endoglycosidases. High mannose and complex oligosaccharides can be differentiated by endoglycosidase H (Endo H) sensitivity, since Endo H will only cleave high mannose glycans. Peptide-N-glycosidase F (PNGase F) will remove all types of N-linked glycosylation indiscriminately. Deglycosylation of eE2 with PNGase F under denatured, reducing conditions resulted in a faster migrating band greater than 31 kDa, consistent with its calculated molecular weight of 33 kDa (FIG. 9A). In contrast, eE2 was largely resistant to digestion with Endo H (FIG. 9A). This result suggests that the majority of the N-linked glycans on eE2 are of the complex form, in accordance with its mode of expression by export through the secretory pathway.

To investigate the glycosylation pattern in further detail, we employed high-resolution mass spectrometry. eE2 was digested with either trypsin or chymotrypsin and samples of the protein fragments were deglycosylated with either PNGase F or Endo H. PNGase F deaminates the asparagine residue to which the N-linked glycan is attached and converts it to aspartic acid. If the glycan is of the high mannose form, it will be sensitive to Endo H, which leaves one N-acetylglucosamine (GlcNAc) bound to the Asn. Thus, the gain of 1 Da (Asn to Asp; nitrogen to oxygen) by PNGase F or 203 Da by the GlcNAc residue left by Endo H cleavage can be resolved by MS of the peptides. From the resulting three spectra (untreated, Endo H and PNGase F treated) we are able to map all the glycosylation sites, estimate the approximate usage of each site, and determine whether the glycan at a particular site was complex or high mannose. We achieved 100% coverage of the eE2 sequence using trypsin and chymotrypsin. Peptides from the 11 predicted N-linked glycosylation sites were shown to be fully glycosylated, since we were unable to detect unglycosylated peptides with Asn residues in them (FIG. 9B, upper spectra). Only one of the 11 glycosylation sites was found to be Endo H sensitive (VGGVEHRLTAACNF, data not shown for Endo H), suggesting that the majority of the glycans are complex in nature (FIG. 9B, lower spectra). Peptides containing the potential O-linked glycosylation sites were resolved and shown to be unmodified (data not shown).

### Example 16

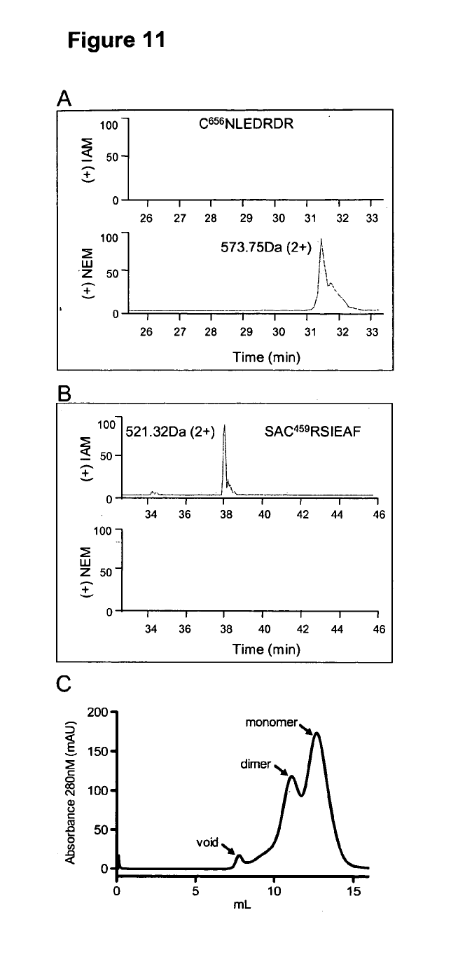

Oligomeric state of eE2. Since previous reports have shown that E2 tends to aggregate, we set out to define the oligomeric state of eE2, using nonreducing SDS-PAGE, size exclusion chromatography and analytical ultracentrifugation. SDS-PAGE analysis of eE2 under non-reducing conditions demonstrated that the eE2 consisted of a mixture of two components with approximate molecular weights of ˜120 kDa (dimer) and ˜60 kDa (monomer) without any large, disulfide-linked aggregates (FIG. 10A). The dimer is formed by an intermolecular disulfide bond, since the addition of reducing agent (β-mercaptoethanol) yielded monomer (FIG. 10A). Size exclusion chromatography of eE2 under native conditions yielded two major peaks and a slight peak found in the void volume of the column. (FIG. 10B). The major and minor peaks were measured at ˜123 kD and ˜75 kDa, respectively, with a significantly greater proportion of dimer. The ratio of dimer to monomer in non-reduced SDS-PAGE (FIG. 10A) and gel filtration (FIG. 10B) is remarkably similar.

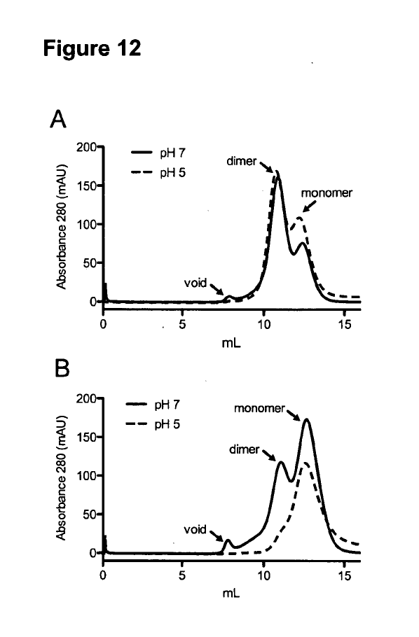

Since, there are 18 conserved cysteines in E2, this could result in the formation of nine disulfide bonds. The eE2 fragment contains only 17 cysteines, leaving at least one unpaired. We therefore sought to determine which cysteine residue was responsible for the intermolecular disulfide bond demonstrated in FIG. 10A. Since eE2 contains a mixture of monomer and dimer, we employed differential cysteine labeling followed by high-resolution mass spectrometry to distinguish free cysteine residues from those involved in disulfide bonding. Briefly, eE2 was incubated with a molar excess of N-ethylmaleimide (NEM, 125Da) under denaturing conditions to label all free cysteine residues. After disulfide bond reduction with DTT, the newly generated free cysteines were alkylated with iodoacetamide (IAM, 57Da). The modified protein was digested with trypsin, deglycosylated with PNGaseF, and the resulting peptides were resolved by MALDI mass spectrometry. All cysteine-containing peptides were identified and only one peptide (C⁶⁵⁶NLEDRDR) was modified by NEM (FIG. 11A). The expected unmodified molecular weight of this peptide is 1020.45 Da, 1077.45 Da if modified by IAM, or 1145.45 Da if modified by NEM. In FIG. 11A, the spectrum shows a 573.75 Da peak corresponding to this peptide modified by NEM and carrying a +2 charge, while no peptide appears at a position corresponding to a modification by IAM (expected ˜538 Da). All other cysteine-containing peptides were shown to have an addition 57 Da, indicating that they were only freed after reduction with DTT. For example, the expected molecular weight of the unmodified SACRSIEAF peptide is 983.46 Da, 1040.46 Da if modified by IAM, or 1108.46 if modified by NEM. This peptide resolves at 521.32 Da, which corresponds to the molecular weight when modified by IAM and carrying a +2 charge. It does not resolve as modified by NEM (expected ˜554 Da) (FIG. 11B).

Consequently, we generated a mutant form of eE2 in which C656 was mutated to a serine (eE2-C656S). Serine was chosen to conserve the biochemical properties at this position. We generated a HEK293T cell line that stably expresses eE2-C656S-Fc and the protein was purified as before. eE2-C656S was analyzed by size exclusion chromatography under conditions identical to the eE2 wild type experiment and the results demonstrated that the mutant was predominantly monomeric at pH7.5 (FIG. 11C).

### Example 17

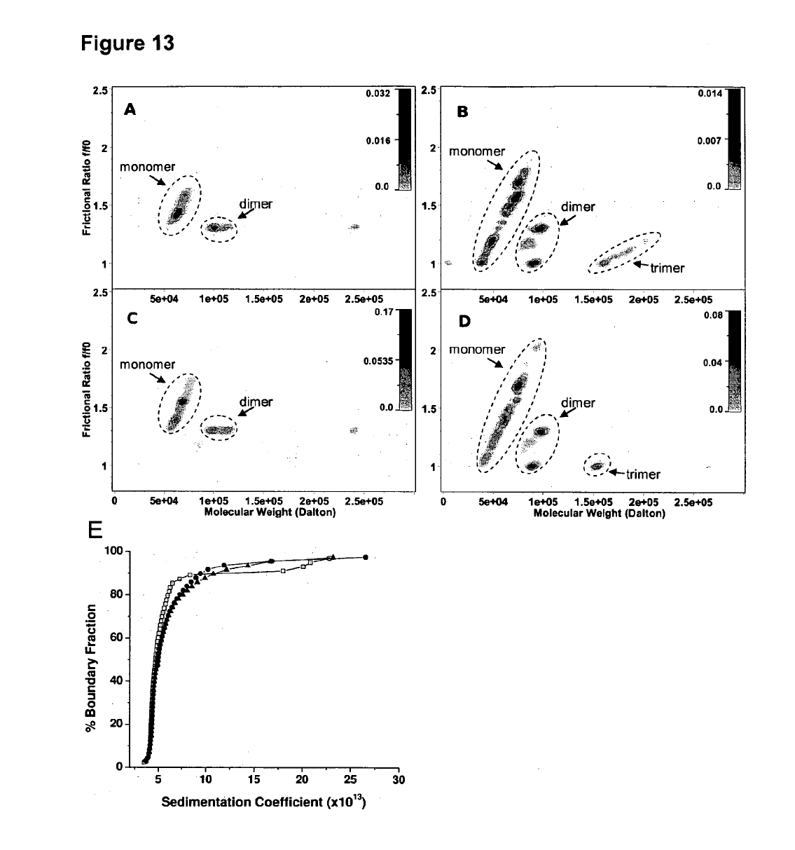

Analysis of eE2 and eE2-C656S at low pH Entry of HCV and other flaviviruses is a pH-triggered event. The single envelope protein (E) of flaviviruses undergoes a slight conformation shift upon incubation at low pH, resulting in a change in the oliogomeric state from dimer at neutral pH to trimer at low pH. Therefore we were interested in determining if HCV eE2 or eE2-C656S undergoes any structural or oligomeric changes when the pH is lowered. Gel filtration results for eE2 at pH 5 revealed a slight increase in the amount of monomer relative to pH 7 (FIG. 12). This was not surprising, since the dimeric form is stabilized by a disulfide bond and should therefore be pH insensitive. Conversely, gel filtration results for eE2-C656S at pH 5 showed an increased proportion of monomer relative to dimer (FIG. 12) and the disappearance of the peak found in the void volume at pH 7. These results provide the first physical evidence of a possible shift in E2 oligomerization in response to low pH.

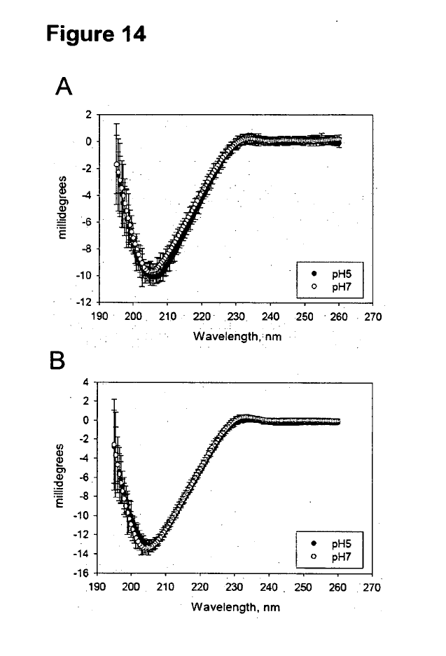

Sedimentation velocity experiments by analytical ultracentrifugation can be used to determine frictional properties (mass and shape) of proteins without the use of standards or interactions with sieving matrix. Sedimentation of eE2 at pH 7 indicates the presence of two dominant species, a monomeric form (60-70 kDa) and a dimeric form (80-130 kDa) (FIG. 13A-D). There is a minor third species that has the approximate molecular weight of a trimer (150-200 kDa). This species seems to correspond to the small peak found in the void volume in size exclusion chromatography. The monomer has a frictional ratio in the range of 1.0-2.0 where a perfect sphere would have a frictional ratio of 1.0. It is interesting to note that the frictional ratio shows a decreasing trend with increasing molecular weight, suggesting a more globular shape for the oligomeric forms. Analysis of eE2 at pH 5 changes the percentage of the three species from 65%:29%:6% to 73%:27%:0% (monomer:dimer:trimer). There is no appreciable difference in the molecular weight or shape distributions at the two concentrations used in this analysis (OD230 nm of 0.25 and 0.8), indicating little or no mass action effects. We also investigated if the shift to lower-molecular weight species at pH 5 is reversible when the pH is restored to 7 (FIG. 13E). It is clearly visible that a drop in pH to 5 reduces the sedimentation speed of about 30-40% of the sample by an appreciable amount, and that this portion is essentially restored to the higher sedimentation coefficient when the sample is returned back to pH 7. This demonstrates that the oligomerization behavior of eE2 when undergoing a pH shift is completely reversible.

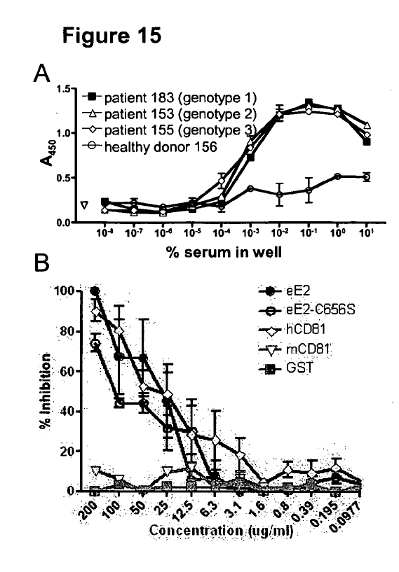

The HCV glycoproteins are predicted to be class II fusion proteins, due to their similarity to alphaviruses and flaviviruses. These proteins are composed of mostly β sheet structure and do not undergo major structural rearrangements upon exposure to low pH. We investigated this possibility by measuring circular dichroism of eE2 and eE2-C656S at pH 7 and pH 5. The resulting spectra revealed that both eE2 and eE2-C656S contain predominantly β-sheet structure and random coil with a small amount of α-helix (FIG. 14). The CD spectra at pH 7 and pH 5 are super-impossible for both constructs, indicating that changes in the oligomeric nature of eE2 by lowering the pH are not due to rearrangement of secondary structure.

### Example 18

### Recognition of J6 eE2

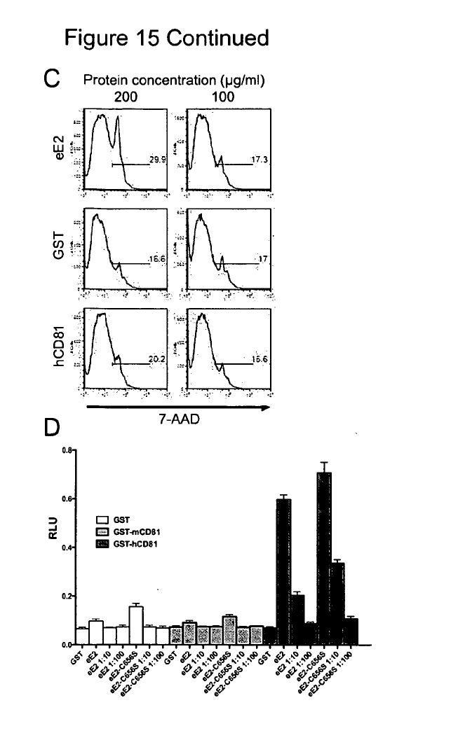

J6 eE2 is recognized by antibodies from patients chronically infected with different genotypes of HCV. The presence of high levels of anti-E2 antibodies in HCV-infected human serum has been reported. In order to further examine if purified eE2 is conformationally similar to the E2 present on infectious HCV particles, we tested whether eE2 was recognized by antibodies from infected patient sera. An enzyme-linked immunosorbent assay (ELISA) plate was coated with eE2 and probed with serum from patients chronically infected with either genotype 1, 2 or 3. Serum from a healthy donor was tested in parallel as a negative control. Anti-human HRP was used to quantify the result. The serum of infected patients bound to eE2 at similar titers regardless of genotype, while the serum of the uninfected donor responded at background levels (FIG. 15A). This illustrates the capacity of eE2 to be recognized by antibodies in patient sera, while also pointing out the maintenance of cross-reactive epitopes.

### Example 19

### Inhibition of Virus Entry

eE2 blocks HCVcc entry. In order to confirm correct folding and function of HCV eE2, we performed a similar assay using cell culture derived HCV (HCVcc). ˜100 TCID50 of HCVcc was incubated with serial 2-fold dilutions of purified eE2, eE2-C656S, GST-human-CD81LEL (hCD81), GST-mouse-CD81LEL (mCD81) or GST. eE2, eE2-C656S, and hCD81 reduced the number of focus forming units (FFU) in a concentration dependent manner, while mCD81 and GST protein had no effect. This experiment yielded a 50% blocking efficiency in the range of 25-150 μg/ml for eE2 and eE2-C656S (FIG. 15B). Thus, HCVcc infection can be effectively blocked by eE2 or eE2-C656S. This further supports the congruence between eE2, eE2-C656S, and full length E2 in the context of virus. In order to rule out the possibility that inhibition of viral infection was due to toxicity, we quantified cell death after incubation with purified protein using fluorescence activated cell sorting analysis (FACS). Cells were incubated for 3 days with 2-fold dilutions of eE2, GST and hCD81 followed by staining with Via-Probe™ to estimate viability. As shown in FIG. 15C, purified eE2 and hCD81 are not toxic at 200 μg/mL (the concentration with the highest level of inhibition).

Inhibition of HCVcc entry by eE2 is thought to occur by sequestering cellular receptors. To confirm this, we analyzed the ability of eE2 to bind hCD81 in vitro. We adapted an ELISA first described by Flint et al for the detection of properly folded E2 based on hCD81 binding. We have obtained identical results using wild type and mutant eE2-Fc supernatants (FIG. 15D) and purified eE2 and eE2-C656S. Plates were coated with GST, mCD81, and hCD81, probed with eE2-Fc or eE2-C656S-Fc cell supernatants, then developed with HRP-conjugated anti-Fc. The assay was executed in triplicate using undiluted cell culture supernatant, supernatant diluted 1:10 in media, or supernatant diluted 1:100 in media. Both eE2-Fc and eE2-C656S-Fc specifically bind hCD81 but not mCD81 or GST alone (FIG. 15D).

Discussion

HCV E1 and E2 are primary determinants of entry and pathogenicity. Deletion mutagenesis has defined the ectodomain of HCV E2 to comprise amino acids 384-664 of E2. Functional and biophysical studies of HCV E2 have been hindered by the formation of mostly aggregated, misfolded material. The eE2 protein produced as described here maintains many of the functionalities associated with E2 found on virions. eE2 can compete with HCVcc to inhibit infection, is recognized by antibodies from chronically infected patients, and can specifically bind the large extracellular loop of human CD81.

All eleven of the predicted N-linked glycosylation sites in E2 are utilized with high efficiency. This is consistent with previous data using HCVpp. Ten of the eleven sites have complex glycans attached, while only the most C-terminal is of the high mannose form. This observation suggests that the glycan at this position is concealed from the modification enzymes in the secretory pathway. This may occur as a result of the dimeric interface formed between two eE2-Fc molecules, or because of steric affects of the Fc tag attached to the C-terminus. As reported by Goffard et al, mutation of the C-terminal glycosylation site does not affect folding, secretion, or E1/E2 heterodimer formation, but does result in less than 50% infectivity when incorporated into HCVpp. Peptides containing the putative O-linked glycosylation sites (FIG. 8A) were not modified, as determined by LC-MS. However, at this time, we cannot rule out that O-linked glycosylation occurs at a low level on eE2 or when expressed as part of the HCV polyprotein.

The HCV glycoproteins, like those from related alphaviruses and flaviviruses, are predicted to be class II fusion proteins. This class of proteins are characterized as mostly beta sheet structures that do not undergo changes in secondary structure upon exposure to low pH. The flavivirus E protein (comprised of three domains) is responsible for receptor binding and membrane fusion. Flaviviruses have icosahedral symmetry, with E arranged as 90 dimers. Cryoelectron microscopy has demonstrated that the E protein lies flat on the surface of the viral lipid bilayer. Upon exposure to low pH there is a slight rotation of domain III, resulting in dissociation of the E dimers and rearrangement into trimers via a monomeric intermediate stage. eE2 appears mostly as a monomer and dimer with a much lower amount of trimer at pH7. Upon exposure to low pH there is an increase in the amount of monomer relative to dimer and trimer. This result represents the first observation of a shift in oligomeric arrangement of the HCV glycoproteins. However, the oligomeric state of E2 may be influenced by the deleted C-terminal portion, the high concentration found on virus particles, and/or heterodimerization with E1. CD spectroscopic analysis of HCV eE2 demonstrated a pronounced minimum at about 203 nm, consistent with a mostly n-sheet protein. Exposure to low pH does not result in a major rearrangement of secondary structure as determined by CD. However, CD cannot rule out the possibility of structural rearrangements that preserve the overall proportion of β-sheet and random coil. These data support the categorization of HCV E2 as a member of the class II fusion proteins.

HCV E2 has 18 highly conserved cysteine residues, although the eE2 construct as defined by Michalak et al has the first 17. The 18^{th} is located between the ectodomain and the C-terminal membrane anchor. Differential labeling of free and disulfide-bonded cysteines has demonstrated the presence of eight disulfide bonds and one free cysteine (C656) in eE2. Mutating C656S did not affect inhibition of HCV entry (FIG. 8B), CD81 binding (FIG. 8D), or binding to HCV patient antibodies (data not shown). Our results are consistent with recently published data, showing that all 18 cysteine residues of E2 are in disulfide bonds and reduction of up to half of these disulfides was compatible with HCV entry as well as antibody and CD81 binding.

Based on the results shown in this study and in accordance with previous studies on the HCV envelope proteins, it is highly possible that E2 may provide an excellent vaccine candidate. Chimpanzees immunized with E1/E2 heterodimeric proteins are protected from infection with low doses of homologous hepatitis C virus. In 1991, Weiner et al identified hypervariable regions in E2, while it was later shown that deletion of hypervariable region 1 attenuated infection in chimpanzees. Youn et al did further work in chimpanzees to show that an E2 antibody response correlates with lower viral titres. Most recently, rodents injected with HCV envelope glycoproteins have been shown to produce antibodies that are broadly cross-reactive in their neutralization properties. The production of large quantities of functional and properly folded E2 ectodomain will aid in our understanding of E2 function as well as assist in designing a vaccine and entry inhibitors.

Other modifications and variations of the specific embodiments of the invention as set forth herein will be apparent to those skilled in the art. The disclosure of all references cited herein are hereby incorporated by reference in their entireties.

1. A construct comprising: the ectodomain of the hepatitis C virus (HCV) E2 sequence and a mammalian expression system therefore comprising the CMV promoter, prolactin signal sequence, the ectodomain of HCV E2 truncated at amino acid 664, a thrombin cleavage site and the human Fc domain.

2. The construct of claim 1 that is specific for the J6 HCV genotype.

3. The construct of claim 1 which is a mutant form of eE2 in which C656 was mutated from a cysteine amino acid to a different amino acid.

4. The construct of claim 3, wherein the different amino acid is serine.

5. An expression system for a construct of any of claims 1-4, which is stably expressed in human embryonic kidney (HEK) 293T cells.

6. A method of producing HCV e2 polypeptide comprising:
(i) providing a construct comprising: the ectodomain of the Hepatitis C virus (HCV) E2 sequence and a mammalian expression system therefore comprising the CMV promoter, prolactin signal sequence, the ectodomain of HCV E2 truncated at amino acid 664, a thrombin cleavage site and the human Fc domain,
(ii) introducing the construct into HEK293T cells,
(iii) selection of cells stably expressing the polypeptide,
(iv) incubating the cells stably expressing the polypeptide in a supernatant, and
(v) recovering and purifying the polypeptide from the supernatant.

7. The method of claim 6 producing about 0.5 to about 15 mg of polypeptide per liter of supernatant.

8. The method of claim 6 producing about 0.5 to about 4 mg of polypeptide per liter of supernatant.

9. The method of claim 6 producing about 0.5 to about 2 mg of polypeptide per liter of supernatant.

10. The method of claim 6 which produces the HCV eE2 HCV J 6 genotype polypeptide.

11. The method of claim 6 wherein the construct is a mutant form of eE2 in which C656 was mutated from a cysteine amino acid to a different amino acid.

12. The method of claim 6, wherein the different amino acid is serine.

13. The method of any of claims 6-12 wherein the incubation occurs in one or more vessels suitable for providing an environment for the cells to express the polypeptide.

14. The method of claim 13 wherein the vessels are in a rotating bottle apparatus.

15. The method of claim 14 wherein the vessel is a bioreactor.

16. The method of any of the claims 6-14 wherein the polypeptide is folded and sequesters human HCV receptor cites.

17. The method of any of any of claims 6-15 wherein the polypeptide wherein the amount of polypeptide in monomer and dimer form exceeds the amount of polypeptide existing in higher orders, and is recognized by antibodies in the sera of patients infected with HCV.

18. The method of any of claims 6 wherein the polypeptide contains 17 preserved cysteine residues.

19. A method of vaccinating a patient comprising administering to a patient in need thereof, a sufficient amount of a polypeptide produced by the method of any of claims 6-18 to produce a strong immune response protecting the patient from future HCV infection.

20. A method of vaccinating a patient chronically infected with HCV comprising administering to a patient in need thereof, a sufficient amount of a polypeptide produced by the method of any of claims 6-18 to produce a more robust immune response.

21. The method of claim 20 further comprising administering an additional therapeutic agent that provides a more robust immune response.

22. A method of inhibiting HCV infection in a human patient comprising administering to a patient in need thereof, a sufficient amount of a polypeptide produced by the method of any of claims 6-18 to effectively block entry into the entry site of human cells.

23. The method of claim 22 wherein the sufficient amount of polypeptide is nontoxic to a human patient.

24. The method of claim 22 wherein a sufficient amount of an active fragment of the polypeptide is administered.

25. A method for detection of antibodies to HCV antibodies in human sera comprising contacting the sera with the polypeptide of any of claims 6-18.

26. A method of producing antibodies comprising introducing the polypeptide of any of claims 6-18 to achieve a response that leads to production of antibodies to said polypeptide.
